(19) United States
(12) Patent Application Publication
(10) Pub. No.: US 2006/0286647 A1
(43) Pub. Date: Dec. 21, 2006

(54) Esters of conjugated unsaturated acids (conjugated acid esters), corresponding methods and use

(76) Inventors: Maxime Allard, Munster (DE); Susanne Neumann, Munster (DE); Dietmar Haring, Schriesheim (DE); Bernhard Hauer, Fussgonheim (DE); Heinz-Peter Rink, Munster (DE)

Correspondence Address: BASF CORPORATION, 1609 BIDDLE AVENUE, WYANDOTTE, MI, 48192, US

(21) Appl. No.: 10/543,796

(22) PCT Filed: Jan. 21, 2004
(86) PCT No.: PCT/EP04/00446

§ 371 Date: May 9, 2006

(30) Foreign Application Priority Data
Feb. 10, 2003 (DE) 10305313.1

(51) Int. Cl. C12P 7/64; C07C 51/347

(52) U.S. Cl. 435/134; 554/126

(57) ABSTRACT

Esters (A) of conjugatedly unsaturated carboxylic acids process for preparing them, and compositions containing said esters, curable with actinic radiation, curable thermally and with actinic radiation or curable oxidatively.

The present invention relates to novel esters of conjugatedly unsaturated acids (conjuenic acid esters), in particular to novel esters of conjugatedly unsaturated fatty acids (conjuenic fatty acid esters).

The present invention also relates to a novel process for preparing conjuenic acid esters, especially conjuenic fatty acid esters.

The present invention further relates to the use of the novel conjuenic acid esters, especially the conjuenic fatty acid esters, as or to prepare compositions curable with actinic radiation.

The present invention relates not least to the use of the novel compositions curable with actinic radiation as coating materials, adhesives and sealants for producing coatings, paint systems, primers, adhesive films, and seals, and also for producing moldings and self-supporting films.

The enzymatic esterification of itaconic acid with fatty acids, such as lauric acid, phenylundecanoic acid and undeceneoic acid, is known from the dissertation entitled “Synthesen und Untersuchungen zum Polymerisationsverhalten von Itaconsäurederivaten” [Syntheses and investigations of the polymerization characteristics of itaconic acid derivatives] by Christine Rüdiger, Department of Natural Sciences II, University of Wuppertal, 1998. The use of the esters as or to prepare compositions which are curable with actinic radiation or both thermally and with actinic radiation or which are air-drying (oxidatively curable) is not described.

For curing with actinic radiation, especially with UV radiation, preference is given to the use of what are termed 100% systems as compositions curable with actinic radiation which are substantially or entirely free from organic solvents and whose ingredients are all, or virtually all, incorporated into the three-dimensional network of the cured compositions which forms as they cure, so that there is no need to incinerate volatile organic compounds. These 100% systems therefore also conform to the strict VOC (volatile organic content) guidelines, whose purpose is avoid the emission of volatile organic compounds, especially solvents.

Despite these substantial advantages, the 100% systems which can be cured with actinic radiation also have certain disadvantages. For instance, their viscosity is frequently too high for the majority of application methods, particularly spray application or roller application. As is known, the viscosity can be lowered by using reactive diluents, such as isobornyl acrylate (cf. also Römpp Online, 2002, “reactive diluents”) or 2-ethylhexyl acrylate (cf. also Römpp Online, 2002, “2-ethylhexyl acrylate”). These reactive diluents are able to give the cured compositions good properties, such as good adhesion and also high water resistance and scratch resistance. At the same time, however, they increase the brittleness of the cured compositions. Since the reactive diluents, on curing, form a particularly close-meshed three-dimensional network, they also lead to an unwanted contraction on polymerization. Numerous known reactive diluents, not least, have a very intense, unpleasant odor to them, leading to a severe odor nuisance accompanying the preparation and application of the corresponding compositions that are curable with actinic radiation.

It is an object of the present invention, therefore, to provide novel, olefinically unsaturated monomers, particularly novel conjuenic acid esters, especially novel conjuenic fatty acid esters, which no longer have the disadvantages of the prior art but which instead effectively lower the viscosity of 100% system so that they are easily applied by the conventional methods.

The novel conjuenic acid esters, especially the conjuenic fatty acid esters, ought to be preparable easily, safely and reproducibly, avoiding the known disadvantages of the thermal processes for preparing olefinically unsaturated esters, such as time-consuming syntheses with the high risk of polymerization as a secondary reaction.

The novel compositions curable with actinic radiation, particularly the novel 100% systems, that are prepared with the aid of the novel conjuenic acid esters, particularly of the novel conjuenic fatty acid esters, ought to be able to be cured not only with actinic radiation but also thermally and/or oxidatively. The cure should be rapid and should not accompanied by a disruptive contraction on polymerization.

The resultant novel cured compositions ought to exhibit excellent adhesion to the conventional substrates, excellent water resistance, excellent scratch resistance, and a high anticorrosion effect. They ought not to tend toward embrittlement.

The invention accordingly provides the novel esters (A) of conjugatedly unsaturated carboxylic acids (conjuenic acid esters) of the general formula I:
XₘYₙ (I),
in which the indices m and n stand for 1 or an integer >1 and the variables X and Y are defined as follows:
X is a radical derived from an olefinically unsaturated carboxylic acid containing one or more than one carboxyl group, 6 to 60 carbon atoms and at least two conjugated double bonds in the molecule (conjuenic acid); and
Y is a monovalent or polyvalent organic radical containing at least one bond which can be activated with actinic radiation;
   with the provisos that
(1) m=1 and Y=monovalent radical if X is derived from a conjuenic acid having more than one carboxyl group in the molecule, and
(2) n=1 and X=derived from a conjuenic acid having one carboxyl group in the molecule if Y=polyvalent radical.

The novel esters (A) of conjugatedly unsaturated carboxylic acids are referred to below as “conjuenic acid esters (A) of the invention”.

The invention also provides the novel process for preparing esters of conjugatedly unsaturated carboxylic acids (conjuenic acid esters) by reacting
(i) at least one olefinically unsaturated carboxylic acid containing one or more than one carboxyl group, 6 to 60 carbon atoms and at least two conjugated double bonds in the molecule (conjuenic acid) or at least one ester of said conjuenic acid with
(ii) at least one hydroxyl-containing compound containing at least one bond which can be activated with actinic radiation in the presence of a catalyst, which involves using as catalyst at least one enzyme which catalyzes the transesterification or esterification and/or at least one organism which catalyzes the transesterification or esterification, said process being referred to below as “process of the invention”.

Further subject matter of the invention will emerge from the description.

In the light of the prior art it was surprising and unforeseeable for the skilled worker that the object on which the present invention was based could be achieved by means of the conjuenic acid esters (A) of the invention and by means of the process of the invention.

In particular it was surprising that by virtue of the conjuenic acid esters (A) of the invention it was possible effectively to lower the viscosity of 100% systems curable with actinic radiation, so that they could be applied readily using the conventional methods.

The conjuenic acid esters (A) of the invention were prepared simply, safely and reproducibly without the known disadvantages of the thermal methods of preparing olefinically unsaturated esters, such as time-consuming syntheses with the high risk of polymerization as a secondary reaction.

The curable compositions of the invention, especially the 100% systems of the invention, that were prepared with the aid of the conjuenic acid esters (A) of the invention were curable not only with actinic radiation but also thermally and/or oxidatively. The cure was rapid and was not accompanied by a disruptive contraction on polymerization. Moreover, there was little or no odor nuisance during the preparation, application or curing of the curable compositions of the invention.

The resultant cured compositions of the invention adhered outstandingly to the conventional substrates and had an excellent water resistance, a high anticorrosion effect, and an excellent scratch resistance. Surprisingly, they showed no tendency toward embrittlement.

The conjuenic acid esters (A) of the invention have the general formula I.

In the general formula I the indices m and n stand for 1 or an integer >1, preferably 1, 2, 3 or 4, more preferably 1, 2 or 3, very preferably 1 or 2, especially 1, with the provisos that
(1) m=1 and Y=monovalent radical, as described below, if X, as described below, is derived from a conjuenic acid having more than one carboxyl group, preferably two, three or four, more preferably two or three and especially two carboxyl groups in the molecule, and
(2) n=1 and X=derived from a conjuenic acid having one carboxyl group in the molecule if Y=polyvalent, preferably di-, tri- or tetravalent, more preferably divalent or trivalent and especially divalent radical.

The variable X stands for a radical derived from a carboxylic acid containing conjugated olefinic unsaturation, or conjuenic acid, containing one carboxyl group or more than one carboxyl group, preferably two, three or four, more preferably two or three and especially two carboxyl groups, 6 to 60, preferably 6 to 40 and especially 6 to 30 carbon atoms, and at least two and in particular from two to six, conjugated double bonds in the molecule. In particular the radical X is derived from a conjuenic acid containing one carboxyl group.

The variable X preferably stands for a radical which is derived from a fatty acid containing conjugated olefinic unsaturation, or conjuenic fatty acid.

The conjuenic fatty acids and their esters, described below, are prepared preferably from olefinically unsaturated fatty acids, such as linoleic acid, linolenic acid or arachidonic acid, whose isolated double bonds are converted into conjugated double bonds under the action of alkali or by a biotechnological method.

The conjuenic fatty acids are conventional products and are sold, for example, under the brand name Isomerginsaure® SF, SY or SK by Harburger Fettchemie or under the brand name Edenor® UKD 6010, 5010 and 5020 by Cognis.

In the general formula I the variable Y stands for a monovalent or polyvalent, preferably di-, tri- or tetravalent, more preferably di- or trivalent and especially divalent radical, but in particular a monovalent organic radical, which contains at least one, especially one, bond which can be activated with actinic radiation. In the context of the present invention actinic radiation means electromagnetic radiation, such as near infrared (NIR) visible light, UV radiation, X-rays and gamma radiation, especially UV radiation, and corpuscular radiation, such as electron beams, proton beams, alpha radiation, beta radiation, and neutron beams, especially electron beams.

The bond which can be activated with actinic radiation becomes reactive when exposed to actinic radiation and, together with other activated bonds of its kind, enters into addition polymerization reactions and/or crosslinking reactions which proceed in accordance with free-radical and/or ionic mechanisms. Examples of suitable bonds are carbon-hydrogen single bonds or carbon-carbon, carbon-oxygen, carbon-nitrogen, carbon-phosphorus or carbon-silicon single bonds or double bonds, or carbon-carbon triple bonds. Of these, the carbon-carbon double bonds and triple bonds are advantageous and are therefore used with preference in accordance with the invention. The carbon-carbon double bonds are especially advantageous, and so are used with particular preference. For the sake of brevity they are referred to below as “double bonds”.

The radical Y is preferably selected from the monovalent radicals of the general formula III: 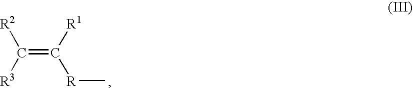
in which the variables are defined as follows:
R is a bonding electron pair between the olefinic carbon atom and the oxygen atom of the oxycarbonyl group of the conjuenic acid radical or organic radical linking to the oxycarbonyl group of the conjuenic acid radical; and
R¹, R²
and R³ independently of one another are each a hydrogen atom or organic radical, it being possible for at least two of the radicals R, R¹, R², and R³ to be cyclically linked to one another;
   and from the polyvalent, preferably di-, tri- and tetravalent, more preferably di- and trivalent and especially divalent radicals of the general formula IV: 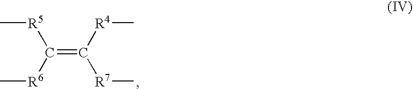
   in which the variables are defined as follows:
R⁴,R⁵,
R⁶ and R⁷ independently of one another are each a bonding electron pair between the olefinic carbon atom and the oxygen atom of the oxycarbonyl group of the conjuenic acid radical or organic radical linking to the oxycarbonyl group of the conjuenic acid radical, with the proviso that a radical R⁴, R⁵, R⁶ or R⁷ which has no linking function is a hydrogen atom or an organic radical, preferably a hydrogen atom, it being possible for at least two of the organic radicals R⁴, R⁵, R⁶ or R⁷ to be linked cyclically to one another.

The linking organic radical R is preferably selected from the group consisting of aliphatic, cycloaliphatic, aromatic, aliphatic-cycloaliphatic, aliphatic-aromatic, cycloaliphatic-aromatic and aliphatic-cycloaliphatic-aromatic radicals which may contain at least one ether, thioether, carboxylate, thiocarboxylate, carbonate, thiocarbonate, phosphate, thiophosphate, phosphonate, thiophosphonate, phosphite, thiophosphite, sulfonate, amide, amine, thioamide, phosphoramide, thiophosphoramide, phosphonamide, thiophosphonamide, sulfonamide, imide, hydrazide, urethane, urea, thiourea, carbonyl, thiocarbonyl, sulfone and/or sulfoxide group.

The linking organic radical R preferably contains at least one carboxylate and/or amide group. With particular preference the divalent organic radical R is composed of a carboxylate group and an alkylene, cycloalkylene and/or arylene group or of an amide group and an alkylene, cycloalkylene and/or arylene group.

Highly suitable alkylene groups R contain one carbon atom or 2 to 6 carbon atoms. Highly suitable cycloalkylene groups R contain 4 to 10, especially 6, carbon atoms. Highly suitable arylene groups R contain 6 to 10, especially 6, carbon atoms.

The variable R stands in particular for a linking organic radical R composed of a carboxylate group and at least one, especially one, alkylene, cycloalkylene and/or arylene group, particularly an alkylene group, (—C(O)—O-alkylene-). With particular preference the variable R stands for the radicals
—C(O)—O—CH₂—,
—C(O)—O—(—CH₂—)₂—,
—C(O)—O—(—CH₂—)₃— and
—C(O)—O—(—CH₂—)₄—.

Examples of suitable organic radicals R¹, R² and R³ include or consist of alkyl, cycloalkyl, and/or aryl groups. Highly suitable alkyl groups contain one carbon atom or 2 to 6 carbon atoms. Highly suitable cycloalkyl groups contain 4 to 10, especially 6, carbon atoms. Highly suitable aryl groups contain 6 to 10, especially 6, carbon atoms.

The organic radicals R, R¹, R² and R³ can be substituted or unsubstituted. However, the substituents must not disrupt the conduct of the process of the invention and/or inhibit the activation of the groups with actinic radiation. Preferably the organic radicals R, R¹, R² and R³ are unsubstituted.

Examples of especially suitable radicals Y of the general formula III are
2-(vinylcarbonyloxy)-,
2-(1-methylvinylcarbonyloxy)-,
2-(1-ethylvinylcarbonyloxy)-,
2-(propenylcarbonyloxy)-,
2-(styrylcarbonyloxy)-,
2-(cyclohexenylcarbonyloxy)-,
2-(endomethylenecyclohexylcarbonyloxy)-,
2-(norbornenylcarbonyloxy)- and
2-(dicyclopentadienylcarbonyloxy)eth-1-yl groups,
3-(1-methylvinylcarbonyloxy)-,
3-(1-ethylvinylcarbonyloxy)-,
3-(propenylcarbonyloxy)-,
3-(styrylcarbonyloxy)-,
3-(cyclohexenylcarbonyloxy)-,
3-(endomethylenecyclohexylcarbonyloxy)-,
3-(norbornenylcarbonyloxy)- and
3-(dicyclopentadienylcarbonyloxy)prop-1-yl groups, and
4-(vinylcarbonyloxy)-,
4-(1-methylvinylcarbonyloxy)-,
4-(1-ethylvinylcarbonyloxy)-,
4-(propenylcarbonyloxy)-,
4-(styrylcarbonyloxy)-,
4-(cyclohexenylcarbonyloxy)-,
4-(endomethylenecyclohexylcarbonyloxy)-,
4-(norbornenylcarbonyloxy)- and
4-(dicyclopentadienylcarbonyloxy)but-1-yl groups, especially
4-(vinylcarbonyloxy)but-1-yl groups.

The linking organic radicals R⁴, R⁵, R⁶ and/or R⁷ are preferably selected from the group consisting of the above-described linking organic radicals R. Particularly advantageous linking organic radicals R⁴, R⁵, R⁶ and/or R⁷ are composed of a carboxylate group and at least one, especially one or two, alkylene, cycloalkylene, and/or arylene groups, in particular one or two alkylene groups, (—C(O)—O-alkylene- or -alkylene-C(O)—O-alkylene-). Especially preferred linking organic radicals R⁴, R⁵, R⁶ and/or R⁷ are
—C(O)—O—CH₂—,
—C(O)—O—(—CH₂—)₂—,
—C(O)—O—(—CH₂—)₃—,
—C(O)—O—(—CH₂—)₄—,
—CH₂—C(O)—O—CH₂—,
—CH₂—C(O)—O—(—CH₂—)₂—,
—CH₂—C(O)—O—(—CH₂—)₃—, and
—CH₂—C(O)—O—(—CH₂—)₄—.

The nonlinking organic radicals R⁴, R⁵, R⁶ and/or R⁷ are preferably selected from the group consisting of the above-described organic radicals R¹, R² and R³.

Examples of especially suitable radicals Y of the general formula IV are
—CH₂—O—(O)C—CH═CH—C(O)—O—CH₂—,
—(—CH₂—)₂—O—(O)C—CH═CH—C(O)—O—(—CH₂—)₂—,
—(—CH₂—)₃—O—(O)C—CH═CH—C(O)—O—(—CH₂—)₃— and
—(—CH₂—)₄—O—(O)C—CH═CFH—C(O)—O—(—CH₂—)₄—
in the cis and trans forms and also 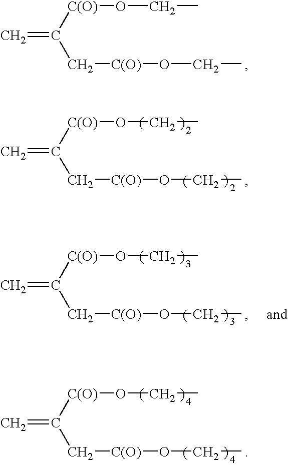
The conjuenic acid esters (A) of the invention may include at least one conjuenic acid ester (B) of the general formula II:
XₘZ (II),
in which the index m stands for 1 or an integer >1, in particular for 1 or 2, especially 2, the variable X is as defined above, and the variable Z stands for a saturated or aromatic, preferably saturated, especially alicyclically saturated, organic radical having a valence of 1 or >1, preferably of 1 or 2, in particular of 2.

Examples of especially suitable monovalent radicals Z are 4-hydroxy-, 3-hydroxy- and 2-hydroxy-cyclohexane and -benzene and also omega-hydroxyalkyl radicals having 1 to 10, preferably 2 to 4, carbon atoms, especially 2-hydroxyethyl, 3-hydroxypropyl and 4-hydroxybutyl radicals.

Examples of especially suitable divalent radicals Z are cyclohexane-1,4-, -1,3- and -1,2-diyl, 1,4-, 1,3- and 1,2-phenylene, and alkylene radicals having 1 to 10, preferably 2 to 4, carbon atoms, especially eth-1,2-ylene, prop-1,3-ylene (trimethylene) and but-1,4-ylene(tetramethylene).

The inventive mixture of conjuenic acid ester (A) of the invention and conjuenic acid ester (B) may contain up to 50% by weight of conjuenic acid ester (B).

The conjuenic acid esters (A) of the invention and the inventive mixture of conjuenic acid ester (A) of the invention and conjuenic acid ester (B) may be prepared basically in accordance with the conventional methods of preparative organic chemistry for the preparation of esters. It is of advantage, however, to prepare them by the process of the invention.

The process of the invention involves reacting
(i) at least one of the above-described conjuenic acids, especially conjuenic fatty acids, or at least one ester of these conjuenic acids, especially conjuenic fatty acids, preferably at least one cycloalkyl, alkyl or aryl ester, preferably an alkyl ester, in particular an ester of a low molecular mass alcohol having 1 to 10, preferably 1 to 4, carbon atoms, especially methanol, ethanol, propanol, isopropanol, n-butanol or isobutanol, or of a polyol, especially ethylene glycol, propylene glycol, neopentyl glycol, glycerol, trimethylolpropane, penta-erythritol or a sugar alcohol such as sorbitol or mannitol,
(ii) with at least one, especially one, hydroxyl-containing compound containing at least one, especially one, of the above-described bonds which can be activated with actinic radiation, in the presence of a catalyst.

In accordance with the invention the catalyst used is at least one, especially one, enzyme which catalyzes the transesterification or esterification and/or at least one, especially one, organism which catalyzes the transesterification or esterification.

Enzymes used are hydrolases [EC 3.x.x.x], especially esterases [EC 3.1.x.x] and proteases [EC 3.4.x.x]. Preference is given to the carboxyl ester hydrolases [EC 3.1.1.x]. Particular preference is given to using lipases as hydrolases. Use is made in particular of lipases from Achromobacter sp., Aspergillus sp., Burkholderia sp., Candida sp., Mucor sp., Penicillium sp., Pseudomonas sp., Rhizopus sp., Thermomyces sp. or porcine pancreas. The enzymes and their functions are described in, for example, Römpp Online 2002, “hydrolases”, “lipases” and “proteases”. They may be mobilized or immobilized.

Suitable organisms include all naturally occurring or genetically modified microorganisms, single-celled life forms or cells which catalyze the transesterification or esterification by means of a hydrolase [EC 3.x.x.x], preferably an esterase [EC 3.1.x.x] or protease [EC 3.4.x.x], with particular preference a carboxyl ester hydrolase [EC 3.1.1.x] and in particular a lipase. It is possible to use all the organisms known to the skilled worker which contain hydrolases. It is preferred to use organisms comprising lipases as hydrolases. Use is made in particular of Achromobacter sp., Aspergillus sp., Burkholderia sp., Candida sp., Mucor sp., Penicillium sp., Pseudomonas sp., Rhizopus sp., Thermomyces sp. and cells from porcine pancreas. The organisms in question may be the unaltered organisms themselves or genetically modified organisms which originally do not express the enzymes, or not to a sufficient extent, and which exhibit a sufficiently high enzyme activity and productivity only following modification. Additionally, the organisms may be adapted by means of genetic modification to the reaction conditions and/or culturing conditions.

The amount of enzyme and/or organism used may vary widely and is guided by the requirements of the case in hand, in particular by the reactivity of the starting products and by the catalytic activity and selectivity of the enzyme or organism, and by the conditions chosen.

The enzyme is used preferably in an amount of from 0.1 to 20%, more preferably from 0.2 to 16%, with particular preference from 0.2 to 14%, with very particular preference from 0.3 to 12% and in particular from 0.5 to 10% by weight, based in each case on the total amount of the starting products.

In the process of the invention it is possible to use any of a wide variety of hydroxyl-containing compounds. What is essential is that during the reaction they provide the above-described radicals Y, especially the radicals Y of the general formulae II or IV. The hydroxyl-containing compounds are preferably selected from the group consisting of carboxylates and carboxamides of the general formulae V to X, more preferably V, VIII, IX and X and especially V: 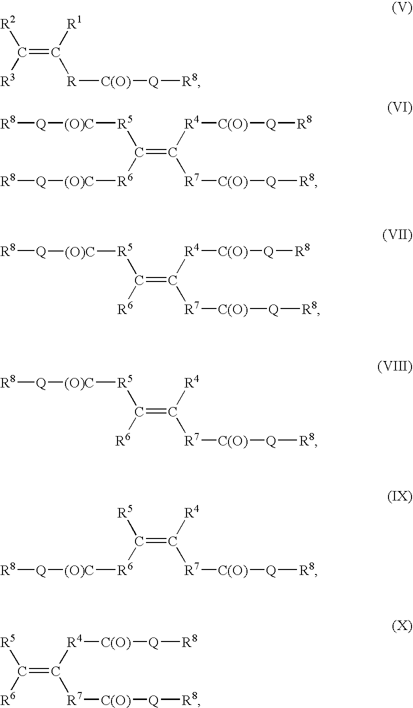
in which the variables R, R¹, R², R³, R⁴, R⁵, R⁶ and R⁷ are as defined above and the variable Q stands for an oxygen atom or a primary or secondary imino group, preferably for an oxygen atom, and the variable R⁸ stands for a hydroxyl-containing monovalent organic radical. The monovalent organic radical R⁸ preferably contains at least one, especially, one, primary and/or secondary, especially primary, hydroxyl group. It also contains at least one, especially one, alkyl, cycloalkyl and/or aryl group, in particular an alkyl group.

The hydroxyl-containing compounds are preferably selected from the group consisting of hydroxyl-containing esters and amides of acrylic acid, methacrylic acid, ethacrylic acid, crotonic acid, cinnamic acid, cyclohexenecarboxylic acid, endomethylenecyclohexanecarboxylic acid, norbornene-carboxylic acid, dicyclopentadienecarboxylic acid, fumaric acid, maleic acid, itaconic acid, endomethylenetetrahydrophthalic acid and methylendomethylenetetrahydrophthalic acid, preferably of acrylic acid, fumaric acid, maleic acid and itaconic acid, in particular of acrylic acid.

Examples of especially suitable hydroxyl-containing compounds are N-methylolacrylamide, 2-hydroxyethyl acrylate, 3-hydroxypropyl acrylate and 4-hydroxybutyl acrylate, especially 4-hydroxybutyl acrylate.

In the process of the invention the molar ratio of conjuenic acid and/or conjuenic acid ester to hydroxyl-containing compound may vary widely. The molar ratio of conjuenic acid and/or conjuenic acid ester to hydroxyl-containing compound is preferably set so as to result in an equivalents ratio of carboxylic acid and/or carboxylate groups to hydroxyl groups of from 0.5:1.0 to 1.0:0.5, more preferably from 0.65:1.0 to 1.0:0.65 and in particular from 0.8:1.0 to 1.0:0.8.

The reactions according to the process of the invention can be carried out in a single-phase or multiphase, aqueous and/or organic reaction medium. The starting products may be present in solution, suspension or emulsion. The reactions can be conducted with or without the addition of solvent. It is preferred to use solvents which are inert with regard to the reactions. Particular preference is given to using conventional organic solvents, especially aprotic nonpolar solvents. It is also possible to use an excess of conjuenic acids and/or conjuenic acid esters or an excess of hydroxyl-containing compounds as the reaction medium. With particular preference the reactions are conducted in bulk, in other words in the absence or the presence of small amounts of organic solvents.

The process of the invention can be conducted at different temperatures. The selection of the temperature range is guided by the requirements of the case in hand, in particular by the reactivity of the starting products and their thermal stability and also by the catalytic activity and selectivity of the enzyme and/or organism and their thermal stability. The process of the invention is preferably conducted at temperatures from 0 to 100° C., more preferably from 10 to 80° C., with particular preference from 15 to 75° C., and in particular from 20 to 70° C.

The duration of the reactions may also vary widely and is likewise guided by the requirements of the case in hand, in particular by the reactivity of the starting products and by the catalytic activity and selectivity of the enzyme and/or organism. The duration is preferably from one hour to one week, more preferably from two hours to five days, with particular preference from three hours to four days, and in particular from four hours to three days.

The process of the invention can be operated in batch mode, in which case all of the starting products are charged to a suitable reaction vessel, or in semibatch mode, in which case some or all of the starting products are metered into the reaction medium during the reaction.

The reactions in accordance with the process of the invention are accompanied by the formation of water or of at least one, especially one, saturated hydroxyl-containing compound, for example methanol, ethanol, propanol or butanol. It is advisable to remove the hydroxyl-containing compound or the water from the reaction mixtures while it is being formed or immediately after it has formed. This can be done employing any conventional method, such as vacuum distillation or azeotropic distillation, pervaporation or passage of inert gases, for example. It is important here that the starting products, the catalysts, and the end products are not damaged thermally. It is also possible to add substances to the reaction mixtures that absorb saturated hydroxyl-containing compounds and/or water. These substances, however, must not disrupt the process of the invention—for example, by lowering the catalytic activity of the enzyme and/or microorganism and/or by developing their own catalytic activity. Examples of suitable absorbents are molecular sieves of appropriate pore size (cf. also Römpp Online, 2002, “molecular sieves” and “zeolites”).

The resultant conjuenic esters (A) of the invention and/or the inventive mixtures of the conjuenic acid esters (A) of the invention and the conjuenic acid esters (B) have numerous special advantages. For instance, they can be crosslinked or cured with just a low dose of actinic radiation, especially UV radiation or electron beams. Further, they can be dried oxidatively, i.e. crosslinked or cured. They are low in odor and do not tend to crystallize out or to form wax. Furthermore, they are highly compatible with all constituents of conventional compositions curable with actinic radiation, oxidatively and/or thermally. In the cured state they have excellent adhesion to metal surfaces.

They can therefore be put to any of a wide variety of end uses. To such ends they can be isolated as substances from the reaction mixtures or used directly in solution. They are preferably used as novel compositions curable with actinic radiation, both thermally and with actinic radiation (dual cure) or oxidatively, or in the function of reactive diluents and/or adhesion promoters for preparing such compositions. The novel compositions curable with actinic radiation, both thermally and with actinic radiation (dual cure) or oxidatively are referred to below as “compositions of the invention”.

The compositions of the invention accordingly contain up to 100% by weight of the conjuenic acid esters (A) of the invention and/or of the inventive mixture of at least one conjuenic acid ester (A) of the invention and at least one conjuenic acid ester (B). The amount in question is preferably from 0.5 to 80%, more preferably from 1 to 60%, with particular preference from 1.5 to 50% and in particular from 2 to 40% by weight, based in each case on the composition of the invention.

The compositions of the invention may comprise any conventional constituents of compositions curable with actinic radiation, oxidatively and/or thermally, such as radiation-curable and thermally curable binders, additional radiation-curable and thermally curable reactive diluents, other than the conjuenic acid esters (A) of the invention, thermally curable reactive diluents, and photoinitiators. They may further include conventional auxiliaries and additives, such as catalysts, plasticizers, light stabilizers, adhesion promoters (tackifiers), slip additives, leveling agents, polymerization inhibitors, flatting agents, nanoparticles, and film-forming auxiliaries.

Examples of suitable conventional constituents of compositions curable with actinic radiation are known for example from the German patent DE 197 09 467 C1, page 4 line 30 to page 6 line 30, or the German patent application DE 199 47 523 A1.

If the composition of the invention is curable thermally as well, i.e. is a dual-cure composition, it preferably further includes conventional thermosetting binders and crosslinking agents, which may also contain groups which can be activated with actinic radiation, and/or thermosetting reactive diluents, as is described in, for example, the German patent applications DE 198 18 735 A1 and DE 199 20 799 A1 or the European patent application EP 0 928 800 A1.

The compositions of the invention are preferably prepared by mixing the above-described constituents in suitable mixing units such as stirred tanks, stirrer mills, extruders, compounders, Ultraturrax devices, inline dissolvers, static mixers, micromixers, toothed-wheel dispersers, pressure release nozzles and/or microfluidizers. It is preferred here to operate in the absence of light with a wavelength λ<550 nm or in complete absence of light, in order to prevent premature crosslinking of the compositions of the invention.

The compositions of the invention may be present in any of a wide variety of forms. Thus, they are conventional compositions containing organic solvents, aqueous compositions, substantially or completely solvent-free and water-free liquid compositions (100% systems), substantially or completely solvent-free and water-free solid powders, or substantially or completely solvent-free powder suspensions (powder slurries). Moreover, they may be one-component systems, in which the binders and the crosslinking agents are present alongside one another, or two-component or multicomponent systems, in which the binders and the crosslinking agents are separate from one another until shortly before application. In particular they are 100% systems.

The compositions of the invention are used for producing compositions cured with actinic radiation, dual-cure compositions and oxidatively cured compositions, preferably coatings, primers, adhesive films, seals, moldings, and self-supporting films, especially primers.

To produce the moldings and films of the invention, the compositions of the invention are applied to conventional temporary or permanent substrates. For producing the films and moldings of the invention it is preferred to use conventional temporary substrates, such as metal belts, plastic belts or hollow bodies made of metal, glass, plastic, wood or ceramic, which can be easily removed without damaging the films and moldings of the invention.

Where the compositions of the invention are used for producing coatings, adhesive films, primers and seals, permanent substrates are employed, such as means of transport, including aircraft, watercraft, rail vehicles, muscle-powered vehicles and motor vehicles, and parts thereof, the interior and exterior of buildings and parts thereof, doors, windows, furniture, hollow glassware, coils, freight containers, packaging, small industrial parts, mechanical components, and components for white goods. The films and moldings of the invention may likewise serve as substrates.

In terms of method, the application of the liquid compositions of the invention has no special features but can instead take place by any conventional application method, such as spraying, squirting, knife coating, brushing, flow coating, dipping, trickling or rolling, for example.

The application of the compositions of the invention in powder form also has no particular features as far as its method is concerned but instead takes place, for example, by the conventional fluid-bed techniques, such as are known, for example, from the BASF Coatings AG brochures “Pulverlacke für industrielle Anwendungen”, January 2000, or “Coatings Partner, Pulverlack Spezial”, 1/2000, or Römpp Lexikon Lacke und Druckfarben, Georg Thieme Verlag, Stuttgart, New York, 1998, pages 187 and 188, “electrostatic powder spraying”, “electrostatic spraying”, and “electrostatic fluidized-bath process”.

During application it is advisable to operate in the absence of actinic radiation in order to prevent premature crosslinking of the compositions of the invention.

The applied compositions of the invention are preferably cured using UV radiation. During irradiation it is preferred to use a radiation dose of from 100 to 6 000, more preferably from 200 to 3 000, more preferably still from 300 to 2 000, and with particular preference from 500 to 1 800 mJ cm⁻², the region <1 700 mJ cm⁻² being especially preferred.

The intensity of radiation may vary widely. It is guided in particular by the radiation dose on the one hand and the irradiation time on the other. For a given radiation dose, the irradiation time is guided by the belt speed or rate of advance of the substrates in the irradiation unit, and vice versa.

As radiation sources for UV radiation it is possible to use all conventional UV lamps. Flash lamps are also suitable. As UV lamps it is preferred to use mercury vapor lamps, more preferably low, medium and high pressure mercury vapor lamps, especially medium pressure mercury vapor lamps. Particular preference is given to using unmodified mercury vapor lamps plus appropriate filters or modified, especially doped, mercury vapor lamps.

Preference is given to using gallium-doped and/or iron-doped, especially iron-doped, mercury vapor lamps, as described for example in R. Stephen Davidson, “Exploring the Science, Technology and Applications of UV and EB Curing”, Sita Technology Ltd., London, 1999, Chapter I, “An Overview”, page 16, FIG. 10, or Dipl.-Ing. Peter Klamann, “eltosch System-Kompetenz, UV-Technik, Leitfaden für Anwender”, page 2, October 1998.

Examples of suitable flash lamps are flash lamps from the company VISIT.

The distance of the UV lamps from the applied compositions of the invention may vary surprisingly widely and can therefore be tailored very effectively to the requirements of the case in hand. The distance is preferably from 2 to 200, more preferably from 5 to 100, with particular preference from 10 to 50, and in particular from 15 to 30 cm. The lamp arrangement may also be adapted to the circumstances of the substrate and the process parameters. In the case of substrates of complex shape, as are envisaged for automobile bodies, those regions not accessible to direct radiation (shadow regions), such as cavities, folds, and other structural undercuts, may be cured using pointwise, small-area or all-round emitters, in conjunction with an automatic movement means for the irradiation of cavities or edges.

Irradiation may be carried out under an oxygen-depleted atmosphere. “Oxygen-depleted” means that the oxygen content of the atmosphere is less than the oxygen content of air (20.95% by volume). In principle the atmosphere may also be oxygen-free—that is, made up of an inert gas. Owing to the absence of the inhibitory effect of oxygen, however, this may cause sharp acceleration of radiation curing, possibly leading to inhomogeneties and stresses in the cured materials of the invention. It is therefore of advantage not to lower the oxygen content of the atmosphere to zero % by volume.

In the case of the applied dual-curable compositions of the invention, the thermal cure may take place, for example, with the aid of a gaseous, liquid and/or solid, hot medium, such as hot air, heated oil or heated rollers, or with the aid of microwave radiation, infrared light and/or near infrared (NIR) light. Heating preferably takes place in a forced-air oven or by irradiation using IR and/or NIR lamps. As in the case of the actinic radiation cure, the thermal cure may also take place in stages. The thermal cure takes place advantageously at temperatures from room temperature to 200° C.

Both the thermal cure and the actinic radiation cure may be carried out in stages. They may follow one another (sequentially) or be simultaneous. In accordance with the invention, sequential curing is of advantage and is therefore used with preference. It is particularly advantageous in this case to carry out the thermal cure after the actinic radiation cure.

The actinic radiation cure and/or the thermal cure may be supplemented or supported by the oxidative cure. The oxidative cure may also be carried out alone with a corresponding composition of the curable compositions of the invention.

The resultant films, moldings, coatings, paint systems, primers, adhesive films, and seals of the invention are outstandingly suitable for the coating, adhesive bonding, sealing, wrapping, and packaging of means of transport, including aircraft, watercraft, rail vehicles, muscle-powered vehicles and motor vehicles, and parts thereof, the interior and exterior of buildings and parts thereof, doors, windows, and furniture, and, in the context of industrial coating, of hollow glassware, coils, freight containers, packaging, small industrial parts, such as nuts, bolts or hubcaps, optical components, electrical components, such as wound goods, including coils and stators and rotors of electric motors, mechanical components, and components for white goods, including household appliances, boilers, and radiators.

In particular, however, the compositions of the invention are used as coating materials for producing coatings, paint systems and primers, preferably for producing primers, in particular for producing primers for producing multicoat color and/or effect electrically conductive, magnetically shielding or fluorescent paint systems, especially multicoat color and/or effect paint systems.

In this utility a further essential advantage of the compositions of the invention is manifested: namely that they provide coatings, paint systems and primers whose coat thickness can be varied very widely. The coat thicknesses are preferably between 2 and 100 μm.

The resultant primers of the invention are the bottommost coats of multicoat paint systems, critically determining the adhesion properties and the corrosion prevention. Consequently, deficiencies in the primers are also manifested to a particularly marked extent and can lead to delamination of the primers and/or of the multicoat paint systems and/or to instances of corrosion in the substrates. However, the primers of the invention have a particularly high adhesive strength both to the substrate and to the overlying coats of the multicoat paint systems. Moreover, they protect metal substrates effectively against corrosion.

The substrates of the invention coated with coatings of the invention, bonded with adhesive films of the invention, sealed with seals of the invention and/or wrapped or packaged with films and/or moldings of the invention therefore have outstanding long-term service properties and a particularly long service life.

### EXAMPLES

### Example 1

The Preparation of a Mixture of a Conjuenic Acid Ester (A) and a Conjuenic Acid Ester (B)

196 parts by weight of Isomergin acid (Isomerginsature®) SF from Harburger Fettchemie, containing 60% by weight of an olefinically unsaturated C18:2 fatty acid having two conjugated double bonds, were mixed with 100.9 parts by weight of 4-hydroxybutyl acrylate (94 percent pure), 7.0 parts by weight of Novozym® 435 (immobilized lipase from Candida antarctica from Novozyme, Denmark), 0.05 part by weight of p-methoxyphenol and 0.01 part by weight of phenothiazine. The resulting reaction mixture was stirred under reduced pressure (5 mbar) at 60° C. for 24 hours. The enzyme was then removed by filtration. This gave 250 parts by weight of a pale yellow, slightly oily liquid. This liquid was able to be supplied without further workup directly for all end uses for which it was envisaged.

Analysis gave the following results:

In ¹H nuclear magnetic resonance spectroscopy in CDCl₃ the signal of the ester group at 4.18 ppm, deriving from 4-hydroxybutyl acrylate, was accompanied by a new signal at 4.08 ppm, which indicated the formation of another ester group. The fatty acid content was determined from the signal of the methyl group at 0.9 ppm. The signal ratio of ester groups to fatty acids indicated a degree of esterification of 97%.

Gel permeation chromatography (UV detector, wavelength 254 nm) produced two main signals of approximately equal size at a number-average molecular weight of 600 daltons and 1 200 daltons. Following fractionation and analysis by mass spectrometry, these were assigned to Isomergin acid 1-butyl-4-acryloyl ester (A) (X—O—(—CH₂—)₄—O—(O)C—CH═CH₂) and 1,4-butanediol diisomergic acid ester (B) (X—O—(—CH₂—)₄—O—X).

### Examples 2a to 2d

The Preparation of a Mixture of a Conjuenic Acid Ester (A) and a Conjuenic Acid Ester (B) with Different Lipases

2a) to 2c)

In each case 5 mmol of Isomergin acid SF (1.4 g) were mixed with 5 mmol of 4-hydroxybutyl acrylate (0.721 g) 50 mg of immobilized enzyme and 1 mg of 3 angstrom molecule sieve and the mixture was shaken at 40° C. for six hours. The immobilized enzyme and the molecular sieve were then removed by filtration, and the oils obtained were then analyzed by ¹H NMR (degree of esterification) and gel permeation chromatography (product distribution).

Example 2a) was carried out using immobilized lipase from Candida antarctica (Novozym® 435), Example 2b) using immobilized lipase from Mucor miehei, and Example 2c) using immobilized lipase from Alcaligenes sp.

The results of the experiments can be found in Table 1.

TABLE 1
The influence of the various lipases on the degree
of esterification and product distribution

| | |
|---|---|
| Degree of | Product distribution: |
| esterification | Conjuenic acid ester |

| Example | (%) | (A) | (B) |
|---|---|---|---|
| 2a) | 96 | 59 | 41 |
| 2b) | 88 | 96 | 4 |
| 2c) | 85 | 94 | 6 |

2d)

1 075.2 g of Edenor® UKG 6010 from Cognis, containing 58 to 63% of an olefinically unsaturated C_{18:2} fatty acid having two conjugated double bonds, were mixed with 553.7 g of 4-hydroxybutyl acrylate, 277 mg of 4-methoxyphenol, 55 mg of phenothiazine and 38.4 g of immobilized lipase from Mucor miehei. The reaction mixture was stirred under reduced pressure (20 to 30 mbar) at 40° C. for eight hours. Ambient air (40 liters/hour) was passed in during this time. The enzyme was then removed by filtration. This gave 1 500 g of a pale yellow, oily liquid which was processed further without additional workup.

¹H nuclear magnetic resonance spectroscopy in CDCl₃ gave a degree of esterification >98% and a product distribution of 93% conjuenic fatty acid ester A and 7% conjuenic fatty acid ester B.

Example 2d) was repeated three times, and the same results were obtained. This underlines the outstanding reproducibility of the process of the invention.

### Examples 3a) to 3c)

### Preparation of a UV-Curable Coating Material and Production of Primers (Examples 3a) and 3b)) and of a Multicoat Paint System (Example 3c)) from it

The coating material was prepared by mixing 100 parts by weight of 3,4-epoxycyclohexylmethyl 3,4-epoxycyclohexanecarboxylate (Cyracure® UVR 6105 from Union Carbide), 0.5 part by weight of a modified silicone (Paint Additive 57 from Dow Coming), 10 parts by weight of 3-ethyl-3-hydroxymethyloxetane (Cyracure® UVR 6000 from Union Carbide), 20 parts by weight of the mixture of the conjuenic acid esters (A) and (B) from Example 1, 4 parts by weight of triarylsulfonium hexafluorophosphate (Cyracure® UVR 6990 from Union Carbide) and 1 part by weight of hydroxycyclohexyl phenyl ketone (Irgacure® 184 from Ciba Specialty Chemicals) and homogenizing the resulting mixture.

The coating material had an advantageously low viscosity and was stable on storage. Even after storage for 1 month in the absence of actinic radiation no instances of phase separation could be observed.

The coating material was applied using a rod-type doctor blade to steel panels of the type HDG (hot-dip galvanized without pretreatment, degreased; Example 3a)) and to steel panels of the type Gardobond 902 (alkali metal phosphatized; Example 3b)) from Chemetall. Following application, the coated steel panels were aged at 50° C. for 12 hours. Application was made in a film thickness such that UV curing with a dose of 1 500 mJ/cm2 resulted in a dry film thickness of from 5 to 10 μm (Example 3a) and from 5 to 6 μm (Example 3b).

The resulting primers of Examples 3a) and 3b) exhibited very good adhesion to the substrate.

In the case of Example 3c) the coating of Example 3b) was overcoated with a conventional pigmented top coat material for industrial applications from BASF Coatings AG. The resultant multicoat paint system exhibited very good adhesion to the substrate and very good intercoat adhesion.

### Examples 4a) and 4b)

Preparation of a UV-Curable Coating Material and Production of Primers from it

A coating material was prepared by mixing 56.7 parts by weight of an epoxy polyether diacrylate (Laromer®V 8986 from BASF Aktiengesellschaft), 0.8 part by weight of cobalt octoate, 10 percent strength in white spirit (Octa Solingen Kobalt 6 in D60 from Borchers GmbH), 18.9 parts by weight of the mixture of the conjuenic acid esters (A) and (B) from Example 1, 18.9 parts by weight of 4-hydroxybutyl acrylate polyphosphoric acid (diphosphorus pentoxide content 84% by weight) in an 80:20 weight ratio and 4.7 parts by weight of hydroxycyclohexyl phenyl ketone (Irgacure® 184 from Ciba Specialty Chemicals) and homogenizing the resulting mixture.

The coating material had an advantageously low viscosity (flow cup, 6 mm nozzle aperture; efflux time 24 s). It was stable on storage: even after storage for 1 month in the absence of actinic radiation it was impossible to observe any instances of phase separation.

The coating material was applied using a rod-type doctor blade to steel panels of type HDG (hot-dip galvanized without pretreatment, degreased) from Chemetall. Following application, the coated steel panels were aged at 50° C. for 12 hours. Application was made in a film thickness such that curing with UV radiation in a dose of 500 mJ/cm² (Example 4a)) and of 1 500 mJ/cm² (Example 4b)) resulted in a dry film thickness of from 5 to 9 μm (Example 4a)) and from 4 to 7 μm (Example 4b)). The resulting primers exhibited very good adhesion to the substrate.

Examples 5a) to 5c) and Comparative Examples C1a) to C1c)

The Preparation of Coating Materials and Production of Primers from them

The coating materials of Examples 5a) to 5c) and of Comparative Examples C1a) to C1c) were prepared by mixing the constituents indicated in Table 2 in the stated amounts and homogenizing the resulting mixture.

TABLE 2
The physical composition of the coating materials of Examples 5a)
to 5c) and of Comparative Examples C1a) to C1c)

| | | | | Comparative | | |
|---|---|---|---|---|---|---|
| | Examples: | | | examples: | | |
| Constituent | 5a) | 5b) | 5c) | C1a) | C1b) | C1c) |
| Laromer ® 8986^{a)} | 60 | 60 | 60 | 60 | 60 | 60 |
| Polyphosphoric ester of 4- | 20 | 20 | 20 | 20 | 20 | 20 |
| hydroxybutyl acrylate^{b)} | | | | | | |
| Irgacure ® 184 | 5 | 5 | 5 | 5 | 5 | 5 |
| Isomergin acid SF 4- | 20 | 40 | 60 | — | — | — |
| hydroxybutyl ester (93 | | | | | | |
| percent pure) | | | | | | |
| 2-ethylhexyl acrylate | — | — | — | 20 | 40 | 60 |
| Cobalt octoate^{c)} | — | 1 | 1 | — | — | — |

^{a)}Epoxy polyether diacrylate from BASF Aktiengesellschaft;
^{b)}Prepared by reacting 80 parts by weight of 4-hydroxybutyl acrylate and 20 parts by weight of polyphosphoric acid with a diphosphorus pentoxide content of 84% by weight;
^{c)}10 percent strength in white spirit.

The coating materials of Examples 5a) to 5c) and of Comparative Examples C1a) were stable on storage in the absence of actinic radiation and showed no instances of clouding or phase separation even after storage for one month. They had an advantageously low viscosity and were easy to apply. The coating materials of Comparative Examples C1b) and C1c) exhibited instances of clouding after a short time, which were attributable to the incompatibility of 2-ethylhexyl acrylate with the other constituents of the coating materials.

The coating materials were applied using rod-type doctor blades to steel panels of type HDG (hot-dip galvanized without pretreatment, degreased) from Chemetall. Following application, the coated steel panels were aged at 50° C. for 12 hours. Application was made in film thicknesses such that curing with UV radiation in a dose of 1 500 mJ/cm² resulted in coatings having dry film thicknesses of from 5 to 9 μm.

UV radiation of the coating materials of Examples 5a) to 5c) was not accompanied by any odor nuisance. In contrast, irradiation of the coating material of Comparative Example C1a) was accompanied by an unpleasant odor. In the case of the coating materials of Comparative Examples C1b) and C1c) an even more intensely pungent odor was produced, leading to a particularly severe odor nuisance. This was attributed to evaporating 2-ethylhexyl acrylate.

The gloss of the coatings was measured in accordance with DIN 67530 at an angle of 60°.

The solvent resistance was determined conventionally by treating the surface of the coatings with a cotton pad soaked with methyl ethyl ketone. The measurements were carried out (i) immediately after the production of the coatings and (ii) after they had been stored at 50° C. for 15 hours. The parameter reported was the number of double rubs after which damage occurred to the surface.

A measurement was also made of the time after which the coatings swelled when kept immersed in methyl ethyl ketone.

The adhesion of the coatings to the substrates was determined by means of the cross-cut test in accordance with DIN ISO 2409:1994-10.

The corrosion control effect was determined by means of the spray mist test in accordance with DIN 53167:1985-12 on scored coatings. In this test a check was made after 72 hours as to whether delamination had occurred (delamination=D) or not (no delamination=nD). After 100 and 120 hours checks were made as to whether marked white corrosion had occurred (white corrosion at the scribe mark >2 mm=W) or minimal or no white corrosion (white corrosion at the scribe mark <1 mm=nW).

The results of the experiments can be found in Table 3.

TABLE 3
Gloss, solvent resistance, adhesion and corrosion control effect of
the coatings of Examples 5a) to 5c) and of Comparative
Examples C1a) to C1c)

| | | Methyl | | | | | | |
|---|---|---|---|---|---|---|---|---|
| | | ethyl | | | | | | |
| | | ketone: | | | Cross- | | | |
| | Gloss | Double rubs | | Swelling | cut | Spray mist testing | | |
| Ex. | (60°) | (i) | (ii) | (min) | testing | 72 h | 100 h | 120 h |
| 5a) | 107 | >100 | >200 | >6 | GT 0 | nD | nW | nW |
| 5b) | 107 | 25-30 | >200 | >4 | GT 0 | D | — | — |
| 5c) | 105 | 15-20 | 180 | 3 | GT 0 | D | — | — |
| C1a) | 107 | >100 | >200 | >6 | GT 0 | nD | nW | nW |
| C1b) | 83 | 15-20 | 180 | 2.25 | GT 0 | D | — | — |
| C1c) | 34 | 10-12 | 80 | 1.75 | GT 1 | D | — | — |

Comparison of the results of the experiments indicated that the conjuenic acid ester (A) was a completely valid substitute for the conventional reactive diluent 2-ethylhexyl acrylate. Furthermore, the conjuenic acid ester (A) afforded the essential advantages that during the preparation of the coatings in question there was no separation of the constituents, no evaporation of monomers and no odor nuisance. The outstanding compatibility of the conjuenic acid ester (A) resulted in an outstanding gloss of the coatings of the inventive examples. In contrast, at relatively high concentrations of 2-ethylhexyl acrylate, there were instances of clouding and a loss of gloss in the coatings of the comparative examples. Moreover, the conjuenic acid ester (A) improved the solvent resistance of the coatings. Not least, it was also possible to carry out subsequent air drying of the coatings of Examples 5b and 5c.

### Example 6 and Comparative Examples C2a) and C2b)

The Preparation of Coating Materials and Production of Primers from them

The coating materials of Example 6 and of Comparative Examples C2a) and C2b) were prepared by mixing the constituents indicated in Table 4 in the stated amounts and homogenizing the resulting mixture.

The coating materials were applied using rod-type doctor blades to degreased bright steel panels from Chemetall. Following application, the coated steel panels were aged at 50° C. for twelve hours. Application was made in film thicknesses such that curing with UV radiation in a dose of 1 500 mJ/cm² resulted in coatings having dry film thicknesses of 8 to 10 μm.

The adhesion of the coatings to the substrates was determined by means of the cross-cut test in accordance with DIN ISO 2409:1994-10.

The corrosion control effect was determined by means of the spray mist test in accordance with DIN 53167:1985-12 on scored coatings. In this test a check was made after 72, 120 and 144 hours as to whether delamination had occurred (delamination=D) or not (no delamination=nD) and as to whether marked corrosion had occurred (corrosion at the scribe mark>2 mm=K) or minimal or no corrosion (corrosion at the scribe mark<1 mm=nK).

The results can likewise be found in Table 4. They demonstrate the outstanding adhesion and very good corrosion control effect of the coating of Example 6.

TABLE 4
The physical composition of the coating materials of Example 6
and of the Comparative Examples C 2a) and C 2b)

| | Examples: | | |
|---|---|---|---|
| Constituents | 6 | C 2a) | C 2b) |
| Phenolic resin (Schenactady SMD 31144), | 12 | — | — |
| 35 percent strength solution in Isomergin | | | |
| acid SF 4-hydroxybutyl ester (93 percent | | | |
| purity) | | | |
| Phenolic resin (Schenactady SMD 31144), | — | 12 | — |
| 35 percent strength solution in 4- | | | |
| hydroxybutyl acrylate | | | |
| Alkyd resin (Alkydal ® R35 from Bayer | 30 | 30 | 42 |
| AG), 35 percent strength solution in | | | |
| isobornyl acrylate | | | |
| N-(2-methacryloyleth-1-yl)ethyleneurea and | 10 | 10 | 10 |
| isobornyl acrylate in a 1:1 weight ratio | | | |
| Isobornyl acrylate | 9.4 | 9.4 | 9.4 |
| Modified polyisocyanate prepolymer based | 17 | 17 | 17 |
| on diphenylmethane diisocyanate | | | |
| (Desmodur ® 2010 from Bayer AG) and 4- | | | |
| hydroxybutyl acrylate | | | |
| Epoxy polyether diacrylate (Laromer ® 8986 | 18 | 18 | 18 |
| from BASF Aktiengesellschaft) | | | |
| Cobalt octoate (10 percent in white spirit) | 0.1 | 0.1 | 0.1 |
| Irgacure ® 184 | 5 | 5 | 5 |
| Adhesion and corrosion control effect: | | | |
| Cross-cut test | GT 0 | GT 0 | GT 0 |
| Spray mist test | | | |
| 72 h | nK, nD | K, D | nK, D^{a)} |
| 120 h | nK, nD | — | K, D |
| 144 h | K, nD^{b)} | — | — |

^{a)}Incipient delamination;
^{b)}Microblisters, 5 mm at the scribe mark.

1. Olefinically unsaturated esters (A) (conjuenic acid esters) of the general formula I:
XₘYₙ (1),
in which the indices m and n stand for 1 or an integer >1 and the variables X and Y are defined as follows:
X is a radical derived from an olefinically unsaturated carboxylic acid containing one or more than one carboxyl group, 6 to 60 carbon atoms and at least two conjugated double bonds in the molecule (conjuenic acid); and
Y is a monovalent or polyvalent organic radical containing at least one bond which can be activated with actinic radiation;
with the provisos that
(1) m=1 and Y=monovalent radical if X is derived from a conjuenic acid having more than one carboxyl group in the molecule, and
(2) n=1 and X=derived from a conjuenic acid having one carboxyl group in the molecule if Y=polyvalent radical.

2. Conjuenic acid esters (A) as claimed in claim 1, further comprising at least one conjuenic acid ester (B) of the general formula II:
XₘZ (II),
in which the index m stands for 1 or an integer >1 and the variables X and Z are defined as follows:
X is a radical derived from a conjuenic acid; and
Z is a hydroxyl-free or hydroxyl-containing, saturated or aromatic organic radical having a valence of ≧1 [or >1].

3. Conjuenic acid esters (A) as claimed in claim 1, wherein the conjuenic acid is a conjugatedly unsaturated fatty acid (conjuenic fatty acid).

4. Conjuenic acid esters (A) as claimed in claim 3, wherein the conjuenic fatty acids are prepared from olefinically unsaturated fatty acids whose isolated double bonds have been converted into conjugated double bonds by exposure to alkali or by a biotechnological method.

5. Conjuenic acid esters (A) as claimed in claim 1, wherein the bonds in the radical Y which are activated with actinic radiation are selected from the group consisting of carbon-carbon double bonds and of carbon-carbon triple bonds and mixtures thereof.

6. Conjuenic acid esters (A) as claimed in claim 5, wherein the bonds in the radical Y which are activated with actinic radiation are carbon-carbon double bonds.

7. Conjuenic acid esters (A) as claimed in claim 5, wherein the radical Y is selected from the monovalent radicals of the general formula III: 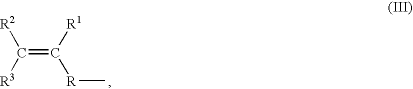
in which the variables are defined as follows:
R is a bonding electron pair between the olefinic carbon atom and the oxygen atom of the oxycarbonyl group of the conjuenic acid radical or organic radical linking to the oxycarbonyl group of the conjuenic acid radical; and
R¹, R²
and R³ independently of one another are each a hydrogen atom or organic radical, it being possible for at least two of the radicals R, R¹, R², and R³ to be cyclically linked to one another;
and from the polyvalent radicals of the general formula IV: 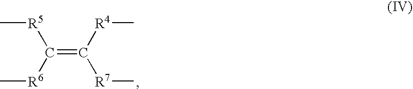
in which the variables are defined as follows:
R⁴, R⁵,
R⁶ and R⁷ independently of one another are each a bonding electron pair between the olefinic carbon atom and the oxygen atom of the oxycarbonyl group of the conjuenic acid radical or organic radical linking to the oxycarbonyl group of the conjuenic acid radical, with the proviso that a radical R⁴, R⁵, R⁶ or R⁷ which has no linking function is a hydrogen atom or an organic radical, it being possible for at least two of the organic radicals R⁴, R⁵, R⁶ or R⁷ to be linked cyclically to one another.

8. Conjuenic acid esters (A) as claimed in claim 7, wherein the nonlinking organic radicals R¹, R², R³, R⁴, R⁵, R⁶ and R⁷ include at least one of alkyl, cycloalkyl and aryl groups.

9. Conjuenic acid esters (A) as claimed in claim 7, wherein the linking organic radicals R, R⁴, R⁵, R⁶ and R⁷ are selected from the group consisting of aliphatic, cycloaliphatic, aromatic, aliphatic-cycloaliphatic, aliphatic-aromatic, cycloaliphatic-aromatic and aliphatic-cycloaliphatic-aromatic radicals which m contain at least one of ether, thioether, carboxylate, thiocarboxylate, carbonate, thiocarbonate, phosphate, thiophosphate, phosphonate, thiophosphonate, phosphite, thiophosphite, sulfonate, amide, amine, thioamide, phosphoramide, thiophosphoramide, phosphonamide, thiophosphonamide, sulfonamide, imide, hydrazide, urethane, urea, thiourea, carbonyl, thiocarbonyl, sulfone and sulfoxide groups.

10. Conjuenic acid esters (A) as claimed in claim 9, wherein the linking organic radicals R, R⁴, R⁵, R⁶ and R⁷ contain at least one of carboxylate groups-and/of amide groups.

11. Conjuenic acid esters (A) as claimed in claim 10, wherein the linking organic radicals R, R⁴, R⁵, R⁶ and R⁷ comprise a carboxylate group and of at least one of alkylene, cycloalkylene and arylene groups.

12. A process for preparing conjuenic acid esters (A) as claimed in claims 1 by reacting
(i) at least one of an olefinically unsaturated carboxylic acid containing one or more carboxyl groups, 6 to 60 carbon atoms and at least two conjugated double bonds in the molecule (conjuenic acid) and one ester of said conjuenic acid with
(ii) at least one hydroxyl-containing compound containing at least one bond which can be activated with actinic radiation
in the presence of a catalyst, wherein the catalyst is at least one of an enzyme which catalyzes the transesterification or esterification at least one organism which catalyzes the transesterification or esterification.

13. The process as claimed in claim 12, wherein the enzyme is selected from the group of the hydrolases [EC 3.x.x.x].

14. The process as claimed in claim 13, wherein the hydrolases [EC 3.x.x.x] are selected from the group consisting of esterases [EC 3.1.x.x] and proteases [EC 3.4.x.x].

15. The process as claimed in claim 14, wherein the hydrolases are carboxyl ester hydrolases [EC 3.1.1.x].

16. The process as claimed in claim 15, wherein the hydrolases are lipases.

17. The process as claimed in claim 16, wherein the lipases are obtained from compounds selected from the group consisting of Achromobacter sp., Aspergillus sp., Burkholderia sp., Candida sp., Mucor sp., Penicillium sp., Pseudomonas sp., Rhizopus sp., Thermomyces sp. and porcine pancreas.

18. The process as claimed in claim 12, wherein the organisms are selected from the group consisting of naturally occurring microorganisms, genetically modified microorganisms, single-celled life forms of and cells which comprise at least one enzyme which catalyzes the transesterification or esterification.

19. The process as claimed in claim 18, wherein the organisms are selected from the group consisting of Achromobacter sp., Aspergillus sp., Burkholderia sp., Candida sp., Mucor sp., Penicillium sp., Pseudomonas sp., Rhizopus sp., Thermomyces sp. and cells from porcine pancreas.

20. The process as claimed in claim 12, wherein the hydroxyl-containing compounds (ii) are selected from the group consisting of carboxylates and carboxamides of the general formula V to X 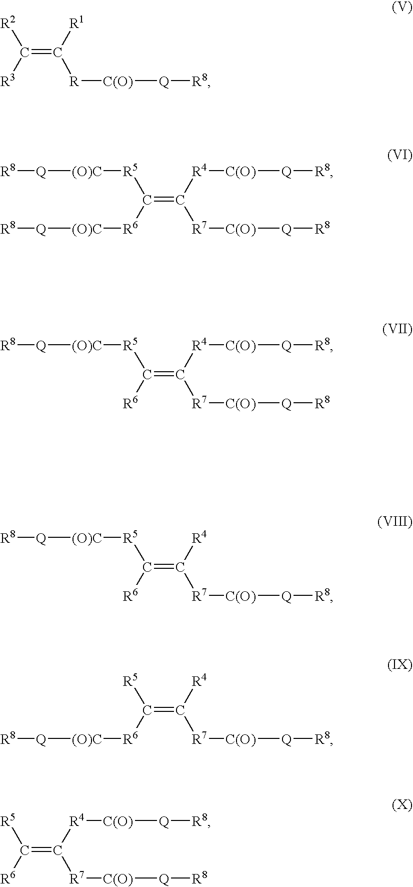
in which the variables R, R¹, R², R³, R⁴, R⁵, R⁶ and R⁷ are as defined above and the variable Q stands for an atom or group selected from the group consisting of an oxygen atom, a primary imino group and a secondary imino group, and the variable R⁸ stands for a hydroxyl-containing monovalent organic radical.

21. The process as claimed in claim 20, wherein the monovalent organic radical R⁸ includes or consists of at least one radical selected from the group consisting of hydroxyl-containing alkyl, cycloalkyl and aryl radicals.

22. The process as claimed in claim 21, wherein the hydroxyl-containing alkyl radical R⁸ is at least one of a hydroxyethyl radical, 2-hydroxyproply radical, 3-hydroxypropyl radical and 4-hydroxybutyl radical.

23. The process as claimed in any of claims 20, wherein the hydroxyl-containing compound (ii) is selected from the group consisting of hydroxyl-containing esters of and hydroxyl-containing amides of at least one of acrylic acid, methacrylic acid, ethacrylic acid, crotonic acid, cinnamic acid, cyclohexene carboxylic acid, endomethylenecyclohexanecarboxylic acid, norbornenecarboxylic acid, dicyclopentadienecarboxylic acid, fumaric acid, maleic acid, itaconic acid, endomethylenetetrahydrophthalic acid and methylendomethylenetetrahydrophthalic acid, maleic acid, fumaric acid and itaconic acid.

24. The process as claimed in claim 22, wherein the hydroxyl-containing compound (ii) is 4-hydroxybutyl acrylate.

25. The process as claimed in claims 21, wherein at least one of the water resulting during the reaction and the hydroxyl-containing compounds resulting during the reaction are removed from the reaction mixture as it or they are formed or immediately after they have been formed.

26. Compositions comprising acid esters (A) as claimed in claim 1 wherein said compositions are curable with at least one of actinic radiation, thermally and oxidatively.

27. Materials selected from the group consisting of coating materials, adhesives, sealants to produce coatings, paint systems, primers, adhesive films, seals, moldings, and self-supporting films comprising the compositions of claim 26.
